SEQUENCE LISTING (3 sequences)

SEQ ID NO: 1 - DNA, 18 bp, Mus musculus
tggagagtgaccggcagc

SEQ ID NO: 2 - DNA, 16 bp, Mus musculus
gccagatgcccgagca

SEQ ID NO: 3 - DNA, 23 bp, Mus musculus
  misc_feature (1)..(1): FAM reporter dye
cctggatgccatgcaggacagct

(19) United States
(12) Patent Application Publication
(10) Pub. No.: US 2008/0107639 A1
(43) Pub. Date: May 8, 2008

(54) Use of a Dg147 Protein Product for Preventing and Treating Metabolic Disorders

(75) Inventor: Daria Onichtchouk, Gottingen (DE)

Correspondence Address: ROTHWELL, FIGG, ERNST & MANBECK, P.C., 1425 K STREET, N.W., SUITE 800, WASHINGTON, DC, 20005, US

(73) Assignee: DEVELOGEN AKTIENGESELLSCHAFT, Goettingen (DE)

(21) Appl. No.: 10/586,671

(22) PCT Filed: Jan. 19, 2005
(86) PCT No.: PCT/EP05/00491

§ 371 Date: Jul. 20, 2006

(30) Foreign Application Priority Data
Jan. 20, 2004 (EP) 04001113.2

(51) Int. Cl. A61K 38/00; A61K 31/70; C12N 5/06; A01K 67/00; G01N 33/567; A61K 35/00; A61P 43/00

(52) U.S. Cl. 424/94.21; 514/12; 514/13; 514/44; 435/375; 800/13; 435/325; 435/366; 435/7.21

(57) ABSTRACT

The present invention discloses novel uses for energy homeostasis regulating proteins and polynucleotides encoding this in the diagnosis, study, prevention, and treatment of metabolic diseases and disorders.

This invention relates to the use of nucleic acid sequences encoding a secreted glycoprotein (DG147), to the use of polynucleotides encoding this, and to the use of modulators/effectors of the protein and polynucleotides in the diagnosis, study, prevention, and treatment of diseases and disorders related to body-weight regulation, for example, but not limited to, metabolic diseases or dysfunctions such as obesity, metabolic syndrome, diabetes mellitus, eating disorder, cachexia, pancreatitis, hypertension, coronary heart disease, hypercholesterolemia, dyslipidemia, osteoarthritis, gallstones, or liver fibrosis.

Most proteins that can be used as a pharmaceutically active compound fall within the family of secreted proteins. Secreted proteins are generally produced within cells at rough endoplasmic reticulum, are then exported to the golgi complex, and then move to secretory vesicles or granules, where they are secreted to the exterior of the cell via exocytosis. Examples for commercially used secreted proteins are human insulin, thrombolytic agents, interferons, interleukins, colony stimulating factors, human growth hormone, transforming growth factor beta, tissue plasminogen activator, erythropoietin, and various other proteins. Receptors of secreted proteins, which are membrane-bound proteins, also have potential as therapeutic or diagnostic agents. It is, therefore, important for developing new pharmaceutical compounds to identify secreted proteins that can be tested for activity in a variety of animal models. Thus, in light of the pervasive role of secreted proteins in human physiology, a need exists for identifying and characterizing novel functions for human secreted proteins and the genes that encode them. This knowledge will allow one to detect, to treat, and to prevent medical diseases, disorders, and/or conditions by using secreted proteins or the genes that encode them.

The pancreas is an essential organ possessing both an exocrine function involved in the delivery of enzymes into the digestive tract and an endocrine function by which various hormones are secreted into the blood stream. The exocrine function is assured by acinar and centroacinar cells that produce various digestive enzymes and intercalated ducts that transport these enzymes in alkaline solution to the duodenum. The functional unit of the endocrine pancreas is the islet of Langerhans. Islets are scattered throughout the exocrine portion of the pancreas and are composed of four cell types: alpha-, beta-, delta- and PP-cells, reviewed for example in Kim S. K. and Hebrok M., (2001) Genes Dev. 15: 111-127. Beta-cells produce insulin, represent the majority of the endocrine cells and form the core of the islets, while alpha-cells secrete glucagon and are located in the periphery. Delta-cells and PP-cells are less numerous and secrete somatostatin and pancreatic polypeptide, respectively.

Early pancreatic development has been well studied in different species, including chicken, zebrafish, and mice (for a detailed review, see Kim & Hebrok, 2001, supra). The pancreas develops from distinct dorsal and ventral anlagen. Pancreas development requires specification of the pancreas anlage along both anterior-posterior and dorsal-ventral axes. A number of transcription factors, which are critical for proper pancreatic development have been identified (see Kim & Hebrok, 2001, supra; Wilson M. E. et al., (2003) Mech Dev. 120: 6580).

In postnatal/adult humans, the acinar and ductal cells retain a significant proliferative capacity that can ensure cell renewal and growth, whereas the islet cells become mostly mitotically inactive. This is in contrast to rodents where beta-cell replication is an important mechanism in the generation of new beta cells. It has been suggested, that during embryonic development, pancreatic islets of Langerhans originate from differentiating duct cells or other cells with epithelial morphology (Bonner-Weir S, and Sharma A, (2002) J. Pathol. 197: 519-526; Gu G. et al., (2003) Mech Dev. 120: 3543). In adult humans, new beta cells arise in the vicinity of ducts (Butler A. E. et al., (2003) Diabetes 52: 102-110; Bouwens L. and Pipeleers D. G., (1998) Diabetologia 41: 629-633). However, also an intra-islet location or an origin in the bone marrow has been suggested for precursor cells of adult beta cells (Zulewski H. et al., (2001) Diabetes 50: 521-533; lanus A. et al., (2003) J Clin Invest. 111: 843-850). Pancreatic islet growth is dynamic and responds to changes in insulin demand, such as during pregnancy or during the increase in body mass occurring during childhood. In adults, there is a good correlation between body mass and islet mass (Yoon K. H. et al., (2003) J Clin Endocrinol Metab. 88: 2300-2308).

Pancreatc beta-cells secrete insulin, which is stimulated by high blood glucose levels. Insulin amongst other hormones plays a key role in the regulation of the fuel metabolism. Insulin leads to the storage of glycogen and triglycerides and to the synthesis of proteins. The entry of glucose into muscles and adipose cells is stimulated by insulin. In patients who suffer from diabetes mellitus type 1 or LADA (latent autoimmue diabetes in adults) (Pozzilli & Di Mario, 2001, Diabetes Care. 8: 1460-1467) beta-cells are being destroyed due to autoimmune attack. The amount of insulin produced by the remaining pancreatic islet cells is too low, resulting in elevated blood glucose levels (hyperglycemia). In diabetes type 2, liver and muscle cells loose their ability to respond to normal blood insulin levels (insulin resistance). High blood glucose levels (and also high blood lipid levels) lead to an impairment of beta-cell function and to an increase in beta-cell apoptosis. It is interesting to note that the rate of beta-cell neogenesis does not appear to change in type 2 diabetics (Butler et al., 2003 supra), thus causing a reduction in total beta-cell mass over time. Eventually the application of exogenous insulin becomes necessary in type 2 diabetics.

Improving metabolic parameters such as blood sugar and blood lipid levels (e.g. through dietary changes, exercise, medication or combinations thereof) before beta cell mass has fallen below a critical threshold leads to a relatively rapid restoration of beta cell function. However, after such a treatment the pancreatic endocrine function would remain impaired due to the only slightly increased regeneration rate.

In type 1 diabetics, the lifespan of pancreatic islets is dramatically shortened due to autoimmune destruction. Treatments have been devised which modulate the immune system and may be able to stop or strongly reduce islet destruction (Raz I. et al., (2001) Lancet 358: 1749-1753; Chatenoud L. et al., (2003) Nat Rev Immunol. 3: 123-132). However, due to the relatively slow regeneration of human beta cells such treatments could only be fully successful at improving the diabetic condition if they are combined with an agent which can stimulate beta cell regeneration.

Thus, both for type 1 and type 2 diabetes (early and late stages) there is a need to find novel agents which stimulate beta cell regeneration.

A variety of model organisms has been used to study the formation of beta cells and to analyze the effect of treatments aimed at the improvement of diabetic conditions. Zebrafish has become a popular model vertebrate for the study of developmental processes as well as for pharmacological and toxicological studies over the last decade (Rubinstein, 2003, Curr Opin Drug Discov Devel. 6(2):218-23; Grunwald & Eisen, 2002, Nat Rev Genet. 3(9): 717-24). In this organism, large numbers of transparent embryos which rapidly develop outside of their mother are readily available. Transgenic lines expressing fluorescent proteins under the control of tissue-specific promoters allow to rapidly assess the effects of pharmacological treatments or gene loss- and gain-of-function treatments. Zebrafish islets contain the same celltypes in a similar spatial organization as mammalian islets. A large number of genes which control pancreatic development in mammals also control pancreatic development in zebrafish (Biemar et al., 2001, Dev Biol. 230(2): 189-203; Ober et al., 2003, Mech Dev. 120(1): 5-18). Suppressing gene function in zebrafish embryos using antisense oligonucleotides, modified Peptide Nucleic Acids (mPNAs) or other antisense compounds with good efficiency and specificity yields phenotypes which are usually indistinguishable from genetic mutants in the same gene (Nasevicius et al., Nat. Genet. 2000 26(2):216-20; Effimov et al., NAR 26; 566-575; Urtishak et al., 5th international conference on zebrafish development and genetics, Madison/WI 2002, abstr. #17). Thus, zebrafish embryos represent a relevant model to identify genes or compounds which control beta cell formation in humans.

Diabetes is a very disabling disease, because today's common anti-diabetic drugs do not control blood sugar levels well enough to completely prevent the occurrence of high and low blood sugar levels. Out of range blood sugar levels are toxic and cause long-term complications like for example renopathy, retinopathy, neuropathy, and peripheral vascular disease. There are also a host of related conditions, such as obesity, hypertension, heart disease, and hyperlipidemia, for which persons with diabetes are at substantially increased risk.

Apart from the impaired quality of life for the patients, the treatment of diabetes and its long-term complications presents an enormous financial burden to our healthcare systems with rising tendency. Thus, for the prevention or treatment of, type 1 and type 2 diabetes as well as for latent autoimmune diabetes in adults (LADA) there is a strong need in the art to identify factors that induce regeneration of pancreatic insulin producing beta-cells. These factors could restore normal function of the endocrine pancreas once its function is impaired or event could prevent the development or progression of diabetes type 1, diabetes type 2, or LADA.

There are several metabolic diseases of human and animal metabolism, e.g., obesity and severe weight loss, that relate to energy imbalance where caloric intake versus energy expenditure is imbalanced. Obesity is one of the most prevalent metabolic disorders in the world. It is still a poorly understood human disease that becomes more and more relevant for western society. Obesity is defined as a body weight more than 20% in excess of the ideal body weight, frequently resulting in a significant impairment of health. Obesity may be measured by body mass index, an indicator of adiposity or fatness. Further parameters for defining obesity are waist circumferences, skinfold thickness and bioimpedance. It is associated with an increased risk for cardiovascular disease, hypertension, diabetes, hyperlipidaemia and an increased mortality rate.

Obesity is influenced by genetic, metabolic, biochemical, psychological, and behavioral factors, and can be caused by different reasons such as non-insulin dependent diabetes, increase in triglycerides, increase in carbohydrate bound energy and low energy expenditure (Kopelman P. G., (2000) Nature 404: 635-643).

The concept of ‘metabolic syndrome’ (syndrome x, insulin-resistance syndrome, deadly quartet) was first described 1966 by Camus and reintroduced 1988 by Reaven (Camus J. P., (1966) Rev Rhum Mal Osteoartic 33: 10-14; Reaven G. M. et al., (1988) Diabetes, 37: 1595-1607). Today metabolic syndrome is commonly defined as clustering of cardiovascular risk factors like hypertension, abdominal obesity, high blood levels of triglycerides and fasting glucose as well as low blood levels of HDL cholesterol. Insulin resistance greatly increases the risk of developing the metabolic syndrome (Reaven G., (2002) Circulation 106: 286-288). The metabolic syndrome often precedes the development of type II diabetes and cardiovascular disease (Lakka H. M. et al., (2002) JAMA 288: 2709-2716). The control of blood lipid levels and blood glucose levels is essential for the treatment of the metabolic syndrome (see, for example, Santomauro A. T. et al., (1999) Diabetes, 48: 1836-1841).

The molecular factors regulating food intake and body weight balance are incompletely understood. Even if several candidate genes have been described which are supposed to influence the homeostatic system(s) that regulate body mass/weight, like leptin or the peroxisome proliferator-activated receptor-gamma co-activator, the distinct molecular mechanisms and/or molecules influencing obesity or body weight/body mass regulations are not known. In addition, several single-gene mutations resulting in obesity have been described in mice, implicating genetic factors in the etiology of obesity (Friedman J. M. and Leibel R. L., (1992), Cell 69: 217-220). In the obese (ob) mouse a single gene mutation (obese) results in profound obesity, which is accompanied by diabetes (Friedman J. M. et. al., (1991) Genomics 11: 1054-1062).

Therefore, the technical problem underlying the present invention was to provide for means and methods for modulating/effecting (pathological) metabolic conditions influencing body-weight regulation and/or energy homeostatic circuits. The solution to said technical problem is achieved by providing the embodiments characterized in the claims. Accordingly, the present invention relates to novel functions of proteins and nucleic acids encoding these in body-weight regulation, energy homeostasis, metabolism, and obesity. The proteins discloses herein and polynucleotides encoding these are thus suitable to investigate metabolic diseases and disorders. Further new compositions are provided that are useful in diagnosis, treatment, and prognosis of metabolic diseases and disorders as described.

So far, it has not been described that the protein of the invention or a homologous protein are involved in the regulation of energy homeostasis and body-weight regulation and related disorders, and thus, no functions in metabolic diseases and dysfunctions and other diseases as listed above have been discussed. In this invention, we demonstrate that the correct gene dose of DG147 is essential for maintenance of energy homeostasis.

Before the present proteins, nucleotide sequences, and methods are described, it is understood that this invention is not limited to the particular methodology, protocols, cell lines, vectors, and reagents described as these may vary. It is also to be understood that the terminology used herein is for the purpose of describing particular embodiments only, and is not intended to limit the scope of the present invention that will be limited only by the appended claims. Unless defined otherwise, all technical and scientific terms used herein have the same meanings as commonly understood by one of ordinary skill in the art to which this invention belongs. Although any methods and materials similar or equivalent to those described herein can be used in the practice or testing of the present invention, the preferred methods, devices, and materials are now described. All publications mentioned herein are incorporated herein by reference for the purpose of describing and disclosing the cell lines, vectors, and methodologies that are reported in the publications which might be used in connection with the invention. Nothing herein is to be construed as an admission that the invention is not entitled to antedate such disclosure.

The present invention discloses that the DG147 protein (herein referred to as “protein of the invention”) is regulating the energy homeostasis and fat metabolism, and polynucleotides, which identify and encode the protein disclosed in this invention. The invention also relates to vectors, host cells, and recombinant methods for producing the polypeptides and polynucleotides of the invention. The invention also relates to the use of these compounds and effectors/modulators thereof, e.g. antibodies, biologically active nucleic acids, such as antisense molecules, RNAi molecules or ribozymes, aptamers, peptides or low-molecular weight organic compounds recognizing said polynulceotides or polypeptides, in the diagnosis, study, prevention, and treatment of metabolic diseases or dysfunctions, including metabolic syndrome, obesity, and/or diabetes as well as related disorders such as eating disorder, cachexia, hypertension, coronary heart disease, hypercholesterolemia, dyslipidemia, osteoarthritis, gallstones, or liver fibrosis.

DG147 has been implicated in numerous processes, including protection from cell death. DG147 is highly expressed in developing pancreas E16-postnatal day 2 in alpha-cells. DG147 could be considered as a growth factor-like molecule stimulating islet-cell proliferation by paracrine action.

So far, it has not been described that the DG147 protein of the invention is involved in the regulation of energy homeostasis and body-weight regulation and related disorders, and thus, no functions in metabolic diseases and dysfunctions have been discussed.

DG147 protein or homologous proteins and nucleic acid molecules coding therefore are obtainable from vertebrate species, e.g. mammals or birds. Particularly preferred are nucleic acids, particularly nucleic acids encoding a human protein as described in Example 2.

The invention particularly relates to a nucleic acid molecule encoding a polypeptide contributing to regulating the energy homeostasis, wherein said nucleic acid molecule comprises
(a) the nucleotide sequence encoding human DG147 nucleic acids, particularly nucleic acids encoding a human protein as described in Example 2, and/or a sequence complementary thereto,
(b) a nucleotide sequence which hybridizes at 50° C. in a solution containing 1×SSC and 0.1% SDS to a sequence of (a),
(c) a sequence corresponding to the sequences of (a) or (b) within the degeneration of the genetic code,
(d) a sequence which encodes a polypeptide which is at least 85%, preferably at least 90%, more preferably at least 95%, more preferably at least 98% and up to 99.6% identical to the amino acid sequence of the DG147 protein, preferably of the human DG147 protein, particularly a human protein as described in Example 2,
(e) a sequence which differs from the nucleic acid molecule of (a) to (d) by mutation and wherein said mutation causes an alteration, deletion, duplication and/or premature stop in the encoded polypeptide or
(f) a partial sequence of any of the nucleotide sequences of (a) to (e) having a length of 15-25 bases, preferably 25-35 bases, more preferably 35-50 bases and most preferably at least 50 bases.

The invention is based on the finding that DG147 and/or homologous proteins and the polynucleotides encoding these, are involved in the regulation of energy homeostasis. The invention describes the use of these compositions for the diagnosis, study, prevention, or treatment of metabolic diseases or dysfunctions, including metabolic syndrome, obesity, and/or diabetes, as well as related disorders as mentioned above.

In this invention, we used a screen for secreted factors expressed in developing mammalian (mouse) pancreas, as described in more detail in the Examples section (see Example 1). This screen identified DG147 as secreted factor expressed in developing mouse pancreas. The present invention describes mammalian DG147 proteins and the polynucleotides encoding these, in particular human DG147, as being involved in the conditions and processes mentioned above.

The function of the DG147 in mammalian metabolism was validated by analyzing the expression of the transcripts in different tissues and by analyzing the role in adipocyte differentiation (see Examples 3 and 4 for more detail). Expression profiling studies confirm the particular relevance of DG147 as regulator of energy metabolism in mammals.

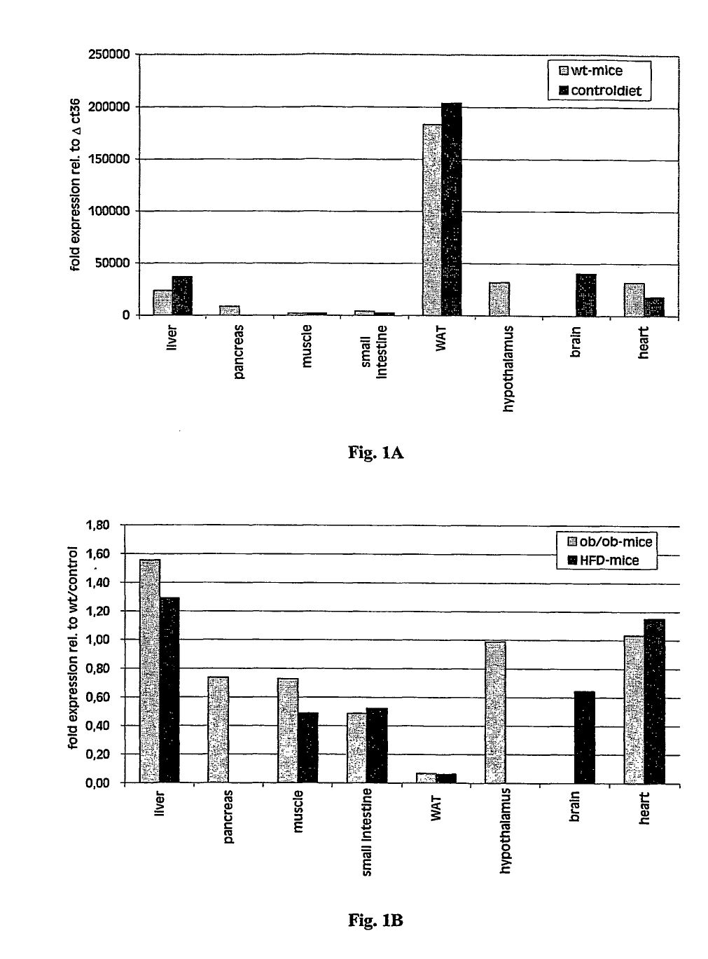

We used mouse models of insulin resistance and/or diabetes, such as mice carrying gene knockouts in the leptin pathway (for example, ob/ob (leptin) or db (leptin receptor/ligand) mice) to study the expression of DG147. Such mice develop typical symptoms of diabetes, show hepatic lipid accumulation and frequently have increased plasma lipid levels (see Bruning J. C. et al., (1998) Mol. Cell. 2: 559-569). Expression of DG147 mRNA was also examined in susceptible wild type mice (for example, C57BI/6) that show symptoms of diabetes, lipid accumulation, and high plasma lipid levels, if fed a high fat diet. DG147 is dramatically down-regulated in the white adipose tissue of both mouse models (see FIG. 1B).

Microarrays are analytical tools routinely used in bloanalysis. A microarray has molecules distributed over, and stably associated with, the surface of a solid support. The term “microarray” refers to an arrangement of a plurality of polynucleotides, polypeptides, antibodies, or other chemical compounds on a substrate. Microarrays of polypeptides, polynucleotides, and/or antibodies have been developed and find use in a variety of applications, such as monitoring gene expression, drug discovery, gene sequencing, gene mapping, bacterial identification, and combinatorial chemistry. One area in particular in which microarrays find use is in gene expression analysis (see Example 4). Array technology can be used to explore the expression of a single polymorphic gene or the expression profile of a large number of related or unrelated genes. When the expression of a single gene is examined, arrays are employed to detect the expression of a specific gene or its variants. When an expression profile is examined, arrays provide a platform for identifying genes that are tissue specific, are affected by a substance being tested in a toxicology assay, are part of a signaling cascade, carry out housekeeping functions, or are specifically related to a particular genetic predisposition, condition, disease, or disorder.

Microarrays may be prepared, used, and analyzed using methods known in the art (see for example, Brennan T. M., (1995) U.S. Pat. No. 5,474,796; Schena M. et al., (1996) Proc. Natl. Acad. Sci. USA 93:10614-10619; Baldeschwieler J. D. et al., (1995) PCT application WO 95/251116; Shalon T. D. and Brown P. O., (1995) PCT application WO 95/35505; Heller R. A. et al., (1997) Proc. Natl. Acad. Sci. USA 94: 2150-2155; Heller, M. J. and Tu E., (1997) U.S. Pat. No. 5,605,662). Various types of microarrays are well known and thoroughly described in Schena M., ed. (1999; DNA Microarrays: A Practical Approach, Oxford University Press, London).

Oligonucleotides or longer fragments derived from any of the polynucleotides described herein may be used as elements on a microarray. The microarray can be used in transcript imaging techniques, which monitor the relative expression levels of large numbers of genes simultaneously as described below. The microarray may also be used to identify genetic variants, mutations, and polymorphisms. This information may be used to determine gene function, to understand the genetic basis of a disorder, to diagnose a disorder, to monitor progression/regression of disease as a function of gene expression, and to develop and monitor the activities of therapeutic agents in the treatment of disease. In particular, this information may be used to develop a pharmacogenomic profile of a patient in order to select the most appropriate and effective treatment regimen for that patient. For example, therapeutic agents, which are highly effective and display the fewest side effects may be selected for a patient based on his/her pharmacogenomic profile.

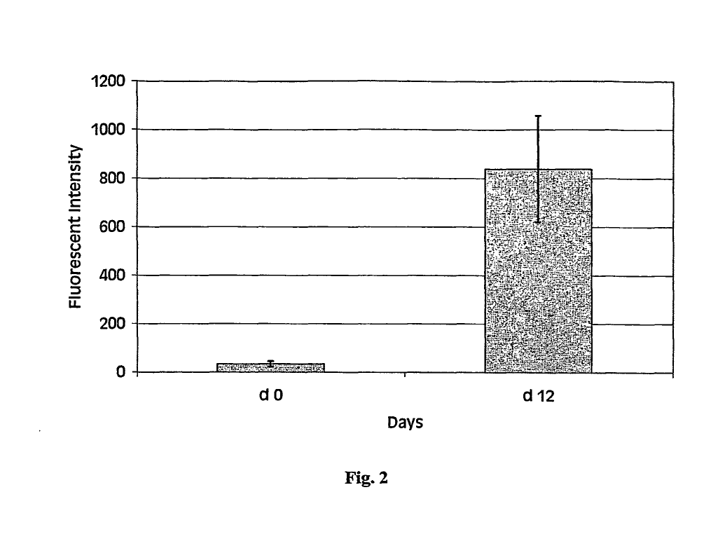

As determined by microarray analysis, DG147 shows differential expression in human primary adipocytes. A strong up-regulation is observed during the human adipocyte differentiation (see FIG. 2). The DG147 protein in preadipocyctes has the potential to enhance adipocyte differentiation at a very early stage. Therefore, the DG147 protein might play an essential role in adipogenesis. The results are suggesting a role of DG147 in the regulation in human metabolism, for example, as effector/modulator (for example, enhancer) of adipogenesis. Thus, DG147 is a strong candidate for the manufacture of pharmaceutical compositions and medicaments for the treatment of conditions related to human metabolism, such as diabetes, obesity, and/or metabolic syndrome.

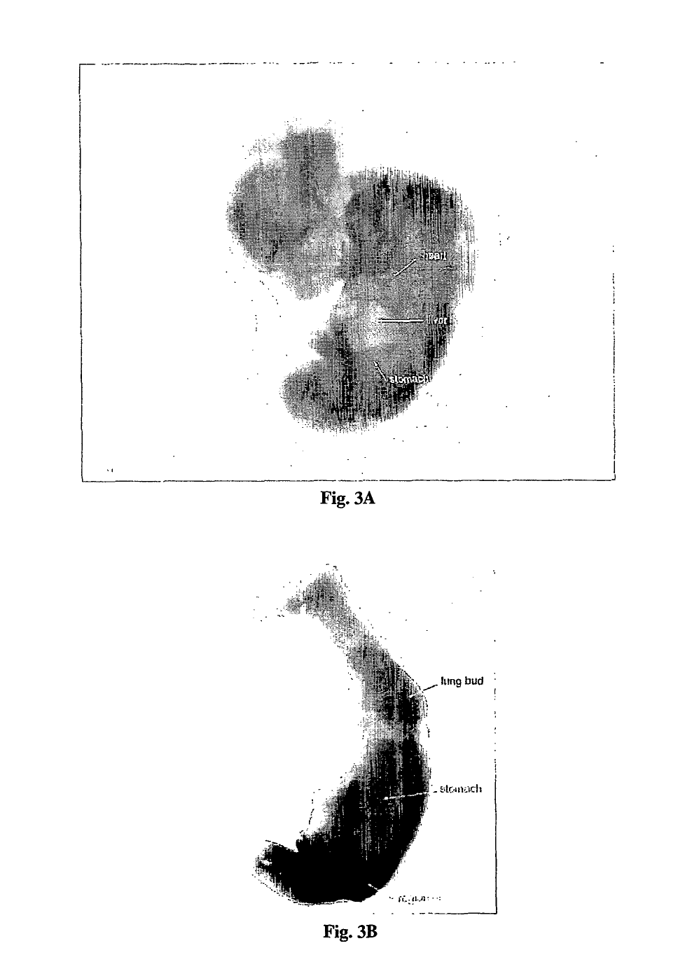

Whole-mount in situ hybridizations were performed according to standard protocols as known to those skilled in the art and as described previously (for example, Pelton, R. W. et al., (1990) Development 110, 609-620; Belo, J. A. et al., (1997) Mech. Dev. 68, 45-57). The nucleic acid sequence encoding the mouse DG147 protein is expressed in the internal organs including the gastrointestinal tract (see FIG. 3).

The invention also encompasses polynucleotides that encode the protein of the invention or a homologous protein. Accordingly, any nucleic acid sequence, which encodes the amino acid sequence of the protein of the invention or a homologous protein, can be used to generate recombinant molecules that express the protein of the invention or a homologous protein. In a particular embodiment, the invention encompasses a nucleic acid encoding human DG147 as described in Example 2; referred to herein as the protein of the invention. It will be appreciated by those skilled in the art that as a result of the degeneracy of the genetic code, a multitude of nucleotide sequences encoding the proteins, some bearing minimal homology to the nucleotide sequences of any known and naturally occurring gene, may be produced. The invention contemplates each and every possible variation of nucleotide sequence that can be made by selecting combinations based on possible codon choices.

Also encompassed by the invention are polynucleotide sequences that are capable of hybridizing to the claimed nucleotide sequences, and in particular, those of the polynucleotides encoding human DG147, or a homologous protein as described in Example 2, under various conditions of stringency. Hybridization conditions are based on the melting temperature (Tm) of the nucleic acid binding complex or probe, as described in Wahl G. M. et al., (1987) Methods Enzymol. 152: 399407 and Kimmel A. R., (1987) Methods Enzymol. 152: 507-511, and may be used at a defined stringency. Preferably, hybridization under stringent conditions means that after washing for 1 h with 1×SSC and 0.1% SDS at 50° C., preferably at 55° C., more preferably at 62° C. and most preferably at 68° C., particularly for 1 h in 0.2×SSC and 0.1% SDS at 50° C., preferably at 55° C., more preferably at 62° C. and most preferably at 68° C., a positive hybridization signal is observed. Altered nucleic acid sequences encoding the proteins which are encompassed by the invention include deletions, insertions or substitutions of different nucleotides resulting in a polynucleotide that encodes the same or a functionally equivalent protein.

The encoded proteins may also contain deletions, insertions or substitutions of amino acid residues, which produce a silent change and result in functionally equivalent proteins. Deliberate amino acid substitutions may be made on the basis of similarity in polarity, charge, solubility, hydrophobicity, hydrophilicity, and/or the amphipathic nature of the residues as long as the biological activity of the protein is retained. Furthermore, the invention relates to peptide fragments of the proteins or derivatives thereof such as cyclic peptides, retro-inverso peptides or peptide mimetics having a length of at least 4, preferably at least 6 and up to 50 amino acids.

Also included within the scope of the present invention are alleles of the genes encoding the protein of the invention or a homologous protein. As used herein, an ‘allele’ or ‘allelic sequence’ is an alternative form of the gene, which may result from at least one mutation in the nucleic acid sequence. Alleles may result in altered mRNAs or polypeptides whose structures or function may or may not be altered. Any given gene may have none, one or many allelic forms. Common mutational changes, which give rise to alleles, are generally ascribed to natural deletions, additions or substitutions of nucleotides. Each of these types of changes may occur alone or in combination with the others, one or more times in a given sequence.

The nucleic acid sequences encoding the protein of the invention or a homologous protein may be extended utilizing a partial nucleotide sequence and employing various methods known in the art to detect upstream sequences such as promoters and regulatory elements. For example, one method which may be employed, ‘restriction-site’ PCR, uses universal primers to retrieve unknown sequence adjacent to a known locus (Sarkar G. et al., (1993) PCR Methods Applic. 2: 318-322). Inverse PCR may also be used to amplify or extend sequences using divergent primers based on a known region (Triglia T. et al., (1988) Nucleic Acids Res. 16: 8186). Another method which may be used is capture PCR which involves PCR amplification of DNA fragments adjacent to a known sequence in human and yeast artificial chromosome DNA (Lagerstrom M. et al., (1991) PCR Methods Applic. 1: 111-119). Another method which may be used to retrieve unknown sequences is that of Parker J. D. et al., (1991) Nucleic Acids Res. 19: 3055-3060. Additionally, one may use PCR, nested primers, and PROMOTERFINDER libraries to walk in genomic DNA (Clontech, Palo Alto, Calif.). This process avoids the need to screen libraries and is useful in finding intron/exon junctions.

In order to express a biologically active protein, the nucleotide sequences encoding the proteins or functional equivalents, may be inserted into appropriate expression vectors, i.e., a vector which contains the necessary elements for the transcription and translation of the inserted coding sequence. Methods, which are well known to those skilled in the art, may be used to construct expression vectors containing sequences encoding the proteins and the appropriate transcriptional and translational control elements. These methods include in vitro recombinant DNA techniques, synthetic techniques, and in vivo genetic recombination. Such techniques are described in Sambrook, J. et al. (1989) Molecular Cloning, A Laboratory Manual, Cold Spring Harbor Press, Plainview, N.Y. and Ausubel, F. M. et al. (1989) Current Protocols in Molecular Biology, John Wiley & Sons, New York, N.Y.

In a further embodiment of the invention, natural, modified or recombinant nucleic acid sequences encoding the protein of the invention or a homologous protein may be ligated to a heterologous sequence to encode a fusion protein. Heterologous sequences are preferably located at the N- and/or C-terminus of the fusion protein.

A variety of expression vector/host systems may be utilized to contain and express sequences encoding the proteins or fusion proteins. These include, but are not limited to, micro-organisms such as bacteria transformed with recombinant bacteriophage, plasmid or cosmid DNA expression vectors; yeast transformed with yeast expression vectors; insect cell systems infected with virus expression vectors (e.g., baculovirus, adenovirus, adeno-associated virus, lentiverus, retrovirus); plant cell systems transformed with virus expression vectors (e.g., cauliflower mosaic virus, CaMV; tobacco mosaic virus, TMV) or with bacterial expression vectors (e.g., Ti or PBR322 plasmids); or animal cell systems.

The presence of polynucleotide sequences encoding the protein of the invention or a homologous protein in a sample can be detected by DNA-DNA or DNA-RNA hybridization or amplification using probes or portions or fragments of said polynucleotides. Nucleic acid amplification based assays involve the use of oligonucleotides or oligomers based on the sequences specific for the gene to detect transformants containing DNA or RNA encoding the corresponding protein. As used herein ‘oligonucleotides’ or ‘oligomers’ refer to a nucleic acid sequence of at least about 10 nucleotides and as many as about 60 nucleotides, preferably about 15 to 30 nucleotides, and more preferably about 20-25 nucleotides, which can be used as a probe or amplimer.

A wide variety of labels and conjugation techniques are known by those skilled in the art and may be used in various nucleic acid and amino acid assays. Means for producing labeled hybridization or PCR probes for detecting polynucleotide sequences include oligo-labeling, nick translation, end-labeling of RNA probes, PCR amplification using a labeled nucleotide, or enzymatic synthesis. These procedures may be conducted using a variety of commercially available kits (Pharmacia & Upjohn, (Kalamazoo, Mich.); Promega (Madison Wis.); and U.S. Biochemical Corp., (Cleveland, Ohio).

Suitable reporter molecules or labels, which may be used for nucleic acid and protein assays, include radionuclides, enzymes, fluorescent, chemiluminescent or chromogenic agents as well as substrates, co-factors, inhibitors, magnetic particles, and the like.

Host cells transformed with nucleotide sequences encoding the protein of the invention may be cultured under conditions suitable for the expression and recovery of said protein from cell culture. The protein produced by a recombinant cell may be secreted or contained intracellularly depending on the sequence and/or the vector used. As will be understood by those of skill in the art, expression vectors containing polynucleotides, which encode the protein may be designed to contain signal sequences, which direct secretion of the protein through a prokaryotic or eukaryotic cell membrane. Other recombinant constructions may be used to join sequences encoding the protein to nucleotide sequence encoding a polypeptide domain, which will facilitate purification of soluble proteins. Such purification facilitating domains include, but are not limited to, metal chelating peptides such as histidine-tryptophan modules that allow purification on immobilized metals, protein A domains that allow purification on immobilized immunoglobulin, and the domain utilized in the FLAG extension/affinity purification system (Immunex Corp., Seattle, Wash.) The inclusion of cleavable linker sequences such as those specific for Factor XA or Enterokinase (Invitrogen, San Diego, Calif.) between the purification domain and the desired protein may be used to facilitate purification.

### Diagnostics and Therapeutics

The data disclosed in this invention show that the nucleic acids and proteins of the invention and effectors/modulators thereof are useful in diagnostic and therapeutic applications implicated, for example but not limited to, metabolic diseases or dysfunctions, including metabolic syndrome, obesity, and/or pancreatic diseases e.g. diabetes mellitus such as insulin dependent diabetes mellitus or non insulin dependent diabetes mellitus, as well as related disorders such as eating disorder, cachexia, hypertension, coronary heart disease, hypercholesterolemia, dyslipidemia, osteoarthritis, gallstones, or liver fibrosis. Particularly, the proteins, nucleic acids and effectors/modulators are useful in applications associated with, accompanied by or caused by disturbances in the differentiation of preadipocytes to adipocytes. Hence, diagnostic and therapeutic uses for the proteins and nucleic acids of the invention are, for example but not limited to, the following: (i) small molecule drug target, (ii) antibody target (therapeutic, diagnostic, drug targeting/cytotoxic antibody), (iii) tissue regeneration in vitro and in vivo (regeneration for all these tissues and cell types composing these tissues and cell types derived from these tissues), (iv) diagnostic and/or prognostic marker, (v) protein therapy, (vi) gene therapy (gene delivery/gene ablation), and (vii) research tools.

The nucleic acids and protein of the invention and modulators/effectors thereof are useful in diagnostic and therapeutic applications implicated in various applications as described below. For example, but not limited to, cDNAs encoding the protein of the invention may be useful in gene therapy, and the protein of the invention may be useful when administered to a subject in need thereof. By way of non-limiting example, the compositions of the present invention will have efficacy for treatment of patients suffering from, for example, but not limited to, metabolic disorders as described above.

The nucleic acids encoding the protein of the invention, a homologous protein, or a functional fragment thereof, may further be useful in diagnostic applications, wherein the presence or amount of the nucleic acids or the proteins are to be assessed. These materials are further useful in the generation of antibodies that bind immunospecifically to the novel substances of the invention may be used in therapeutic or diagnostic methods.

For example, in one aspect, antibodies, which are specific for the protein of the invention or a homologous protein, may be used directly as a modulator/effector, e.g. an antagonist or agonist or indirectly as a targeting or delivery mechanism for bringing a pharmaceutical agent to cells or tissue which express the protein. The antibodies may be generated using methods that are well known in the art. Such antibodies may include, but are not limited to, polyclonal, monoclonal, chimeric single chain, Fab fragments, and fragments produced by a Fab expression library. Neutralising antibodies, (i.e., those which inhibit dimer formation) are especially preferred for therapeutic use.

For the production of antibodies, various hosts including goats, rabbits, rats, mice, humans, and others, may be immunized by injection with the protein or any fragment or oligopeptide thereof which has immunogenic properties. Depending on the host species, various adjuvants may be used to increase immunological response. It is preferred that the peptides, fragments or oligopeptides used to induce antibodies to the protein have an amino acid sequence consisting of at least five amino acids, and more preferably at least 10 amino acids.

Monoclonal antibodies to the proteins may be prepared using any technique that provides for the production of antibody molecules by continuous cell lines in culture. These include, but are not limited to, the hybridoma technique, the human B-cell hybridoma technique, and the EBV-hybridoma technique (Köhler G. and Milstein C. (1975) Nature 256: 495-497; Kozbor D. et al. (1985) J. Immunol. Methods 81: 3142; Cote R. J. et al., (1983) Proc. Natl. Acad. Sci. 80: 2026-2030; Cole S. P. et al., (1984) Mol Cell Biochem. 62: 109-120).

In addition, techniques developed for the production of ‘chimeric antibodies’, the splicing of mouse antibody genes to human antibody genes to obtain a molecule with appropriate antigen specificity and biological activity can be used (Morrison S. L. et al., (1984) Proc. Natl. Acad. Sci. 81: 6851-6855; Neuberger M. S. et al (1984) Nature 312: 604-608; Takeda S. et al., (1985) Nature 314: 452-454). Alternatively, techniques described for the production of single chain antibodies may be adapted, using methods known in the art, to produce single chain antibodies specific for the protein of the invention or a homologous protein. Antibodies with related specificity, but of distinct idiotypic composition, may be generated by chain shuffling from random combinatorial immunoglobulin libraries (Kang A. S. et al., (1991) Proc. Natl. Acad. Sci. 88: 11120-11123). Antibodies may also be produced by inducing in vivo production in the lymphocyte population or by screening recombinant immunoglobulin libraries or panels of highly specific binding reagents as disclosed in the literature (Orlandi R. et al., (1989) Proc. Natl. Acad. Sci. 86: 3833-3837; Winter G. and Milstein C., (1991) Nature 349: 293-299).

Antibody fragments which contain specific binding sites for the proteins may also be generated. For example, such fragments include, but are not limited to, the F(ab′)₂ fragments which can be produced by Pepsin digestion of the antibody molecule and the Fab fragments which can be generated by reducing the disulfide bridges of F(ab′)₂ fragments. Alternatively, Fab expression libraries may be constructed to allow rapid and easy identification of monoclonal Fab fragments with the desired specificity (Huse W. D. et al., (1989) Science 246:1275-1281).

Various immunoassays may be used for screening to identify antibodies having the desired specificity. Numerous protocols for competitive binding and immunoradiometric assays using either polyclonal or monoclonal antibodies with established specificities are well known in the art. Such immunoassays typically involve the measurement of complex formation between the protein and its specific antibody. A two-site, monoclonal-based immunoassay utilizing monoclonal antibodies reactive to two non-interfering protein epitopes are preferred, but a competitive binding assay may also be employed (Maddox D. E. et al., (1983) J. Exp. Med. 158: 1211-1216).

In another embodiment of the invention, the polynucleotides of the invention or fragments thereof or nucleic acid modulator/effector molecules such as aptamers, antisense molecules, RNAi molecules, or ribozymes may be used for therapeutic purposes. In one aspect, aptamers, i.e. nucleic acid molecules, which are capable of binding to the protein of the invention and modulating its activity, may be generated by a screening and selection procedure involving the use of combinatorial nucleic acid libraries.

In a further aspect, antisense molecules may be used in situations in which it would be desirable to block the transcription of the mRNA. In particular, cells may be transformed with sequences complementary to polynucleotides encoding the protein of the invention or a homologous protein. Thus, antisense molecules may be used to modulate/effect protein activity or to achieve regulation of gene function. Such technology is now well known in the art, and sense or antisense oligomers or larger fragments, can be designed from various locations along the coding or control regions of sequences encoding the proteins. Expression vectors derived from retroviruses, adenovirus, herpes or vaccinia viruses or from various bacterial plasmids may be used for delivery of nucleotide sequences to the targeted organ, tissue or cell population. Methods, which are well known to those skilled in the art, can be used to construct recombinant vectors, which will express antisense molecules complementary to the polynucleotides of the genes encoding the protein of the invention or a homologous protein. These techniques are described both in Sambrook et al. (supra) and in Ausubel et al. (supra). Genes encoding the protein of the invention or a homologous protein can be turned off by transforming a cell or tissue with expression vectors, which express high levels of polynucleotides that encode the protein of the invention or a homologous protein or a functional fragment thereof. Such constructs may be used to introduce untranslatable sense or antisense sequences into a cell. Even in the absence of integration into the DNA, such vectors may continue to transcribe RNA molecules until they are disabled by endogenous nucleases. Transient expression may last for a month or more with a non-replicating vector and even longer if appropriate replication elements are part of the vector system.

As mentioned above, modifications of gene expression can be obtained by designing antisense molecules, e.g. DNA, RNA or PNA, to the control regions of the genes encoding the protein of the invention or a homologous protein, i.e., the promoters, enhancers, and introns. Oligonucleotides derived from the transcription initiation site, e.g., between positions −10 and +10 from the start site, are preferred. Similarly, inhibition can be achieved using “triple helix” base-pairing methodology. Triple helix pairing is useful because it cause inhibition of the ability of the double helix to open sufficiently for the binding of polymerases, transcription factors or regulatory molecules. Recent therapeutic advances using triplex DNA have been described in the literature (Gee, J. E. et al. (1994) In; Huber, B. E. and B. I. Carr, Molecular and Immunologic Approaches, Futura Publishing Co., Mt. Kisco, N.Y.). The antisense molecules may also be designed to block translation of mRNA by preventing the transcript from binding to ribosomes.

Ribozymes, enzymatic RNA molecules, may also be used to catalyze the specific cleavage of RNA. The mechanism of ribozyme action involves sequence-specific hybridization of the ribozyme molecule to complementary target RNA, followed by endonucleolytic cleavage. Examples, which may be used, include engineered hammerhead motif ribozyme molecules that can be specifically and efficiently catalyze endonucleolytic cleavage of sequences encoding the protein of the invention or a homologous protein. Specific ribozyme cleavage sites within any potential RNA target are initially identified by scanning the target molecule for ribozyme cleavage sites which include the following sequences: GUA, GUU, and GUC. Once identified, short RNA sequences of between 15 and 20 ribonucleotides corresponding to the region of the target gene containing the cleavage site may be evaluated for secondary structural features which may render the oligonucleotide inoperable. The suitability of candidate targets may also be evaluated by testing accessibility to hybridization with complementary oligonucleotides using ribonuclease protection assays.

Nucleic acid effector/modulator molecules, e.g. antisense molecules and ribozymes of the invention may be prepared by any method known in the art for the synthesis of nucleic acid molecules. These include techniques for chemically synthesizing oligonucleotides such as solid phase phosphoramidite chemical synthesis. Alternatively, RNA molecules may be generated by in vitro and in vivo transcription of DNA sequences. Such DNA sequences may be incorporated into a variety of vectors with suitable RNA polymerase promoters such as T7 or SP6. Alternatively, these cDNA constructs that synthesize antisense RNA constitutively or inducibly can be introduced into cell lines, cells or tissues. RNA molecules may be modified to increase intracellular stability and half-life. Possible modifications include, but are not limited to, the addition of flanking sequences at the 5′ and/or 3′ ends of the molecule or modifications in the nucleobase, sugar and/or phosphate moieties, e.g. the use of phosphorothioate or 2′O-methyl rather than phosphodiesterase linkages within the backbone of the molecule. This concept is inherent in the production of PNAs and can be extended in all of these molecules by the inclusion of non-traditional bases such as inosine, queosine, and wybutosine, as well as acetyl-, methyl-, thio-, and similarly modified forms of adenine, cytidine, guanine, thymine, and uridine which are not as easily recognized by endogenous endonucleases.

Many methods for introducing vectors into cells or tissues are available and equally suitable for use in vivo, in vitro, and ex vivo. For ex vivo therapy, vectors may be introduced into stem cells taken from the patient and clonally propagated for autologous transplant back into that same patient. Delivery by transfection and by liposome injections may be achieved using methods, which are well known in the art. Any of the therapeutic methods described above may be applied to any suitable subject including, for example, mammals such as dogs, cats, cows, horses, rabbits, monkeys, and most preferably, humans.

An additional embodiment of the invention relates to the administration of a pharmaceutical composition, in conjunction with a pharmaceutically acceptable carrier, for any of the therapeutic effects discussed above. Such pharmaceutical compositions may consist of the nucleic acids or protein of the invention or homologous nucleic acids or proteins, antibodies to the protein of the invention or a homologous protein, mimetics, agonists, antagonists or inhibitors of the protein of the invention or homologous proteins or nucleic acids. The compositions may be administered alone or in combination with at least one other agent, such as stabilizing compound, which may be administered in any sterile, biocompatible pharmaceutical carrier, including, but not limited to, saline, buffered saline, dextrose, and water. The compositions may be administered to a patient alone or in combination with other agents, drugs or hormones. The pharmaceutical compositions utilized in this invention may be administered by any number of routes including, but not limited to, oral, intravenous, intramuscular, intra-arterial, intramedullary, intrathecal, intraventricular, transdermal, subcutaneous, intraperitoneal, intranasal, enteral, topical, sublingual or rectal means.

In addition to the active ingredients, these pharmaceutical compositions may contain suitable pharmaceutically-acceptable carriers comprising excipients and auxiliaries, which facilitate processing of the active compounds into preparations, which can be used pharmaceutically. Further details on techniques for formulation and administration may be found in the latest edition of Remington's Pharmaceutical Sciences (Maack Publishing Co., Easton, Pa.).

The pharmaceutical compositions of the present invention may be manufactured in a manner that is known in the art, e.g., by means of conventional mixing, dissolving, granulating, dragee-making, levigating, emulsifying, encapsulating, entrapping or lyophilizing processes. The pharmaceutical composition may be provided as a salt and can be formed with many acids. After pharmaceutical compositions have been prepared, they can be placed in an appropriate container and labeled for treatment of an indicated condition. For administration of proteins, such labeling would include amount, frequency, and method of administration.

Pharmaceutical compositions suitable for use in the invention include compositions wherein the active ingredients are contained in an effective amount to achieve the intended purpose. The determination of an effective dose is well within the capability of those skilled in the art. For any compounds, the therapeutically effective dose can be estimated initially either in cell culture assays, e.g., of preadipocyte cell lines or in animal models, usually mice, rabbits, dogs or pigs. The animal model may also be used to determine the appropriate concentration range and route of administration. Such information can then be used to determine useful doses and routes for administration in humans. A therapeutically effective dose refers to that amount of active ingredient, for example the nucleic acid or protein of the invention or a functional fragment thereof, or an antibody, which is sufficient for treating a specific condition. Therapeutic efficacy and toxicity may be determined by standard pharmaceutical procedures in cell cultures or experimental animals, e.g., ED50 (the dose therapeutically effective in 50% of the population) and LD50 (the dose lethal to 50% of the population). The dose ratio between therapeutic and toxic effects is the therapeutic index, and it can be expressed as the ratio, LD50/ED50. Pharmaceutical compositions, which exhibit large therapeutic indices, are preferred. The data obtained from cell culture assays and animal studies is used in formulating a range of dosage for human use. The dosage contained in such compositions is preferably within a range of circulating concentrations that include the ED50 with little or no toxicity. The dosage varies within this range depending upon the dosage from employed, sensitivity of the patient, and the route of administration. The exact dosage will be determined by the practitioner, in light of factors related to the subject that requires treatment. Dosage and administration are adjusted to provide sufficient levels of the active moiety or to maintain the desired effect. Factors, which may be taken into account, include the severity of the disease state, general health of the subject, age, weight, and gender of the subject, diet, time and frequency of administration, drug combination(s), reaction sensitivities, and tolerance/response to therapy. Long-acting pharmaceutical compositions may be administered every 3 to 4 days, every week or once every two weeks depending on half-life and clearance rate of the particular formulation. Normal dosage amounts may vary from 0.1 to 100,000 micrograms, up to a total dose of about 1 g, depending upon the route of administration. Guidance as to particular dosages and methods of delivery is provided in the literature and generally available to practitioners in the art. Those skilled in the art employ different formulations for nucleotides than for proteins or their inhibitors. Similarly, delivery of polynucleotides or polypeptides will be specific to particular cells, conditions, locations, etc.

In another embodiment, antibodies which specifically bind to the proteins may be used for the diagnosis of conditions or diseases characterized by or associated with over- or under-expression of the protein of the invention or a homologous protein or in assays to monitor patients being treated with the protein of the invention or a homologous protein, or modulators/effectors thereof, e.g. agonists, antagonists, or inhibitors. Diagnostic assays include methods which utilize the antibody and a label to detect the protein in human body fluids or extracts of cells or tissues. The antibodies may be used with or without modification, and may be labeled by joining them, either covalently or non-covalently, with a reporter molecule. A wide variety of reporter molecules which are known in the art may be used several of which are described above.

A variety of protocols including ELISA, RIA, and FACS for measuring proteins are known in the art and provide a basis for diagnosing altered or abnormal levels of gene expression. Normal or standard values for gene expression are established by combining body fluids or cell extracts taken from normal mammalian subjects, preferably human, with antibodies to the protein under conditions suitable for complex formation. The amount of standard complex formation may be quantified by various methods, but preferably by photometry, means. Quantities of protein expressed in control and disease, samples from biopsied Ussues are compared with the standard values. Deviation between standard and subject values establishes the parameters for diagnosing disease.

In another embodiment of the invention, the polynucleotides specific for the protein of the invention or a homologous protein may be used for diagnostic purposes. The polynucleotides, which may be used, include oligonucleotide sequences, antisense RNA and DNA molecules, and PNAs. The polynucleotides may be used to detect and quantitate gene expression in biopsied tissues in which gene expression may be correlated with disease. The diagnostic assay may be used to distinguish between absence, presence, and excess gene expression, and to monitor regulation of protein levels during therapeutic intervention.

In one aspect, hybridization with probes which are capable of detecting polynucleotide sequences, including genomic sequences, encoding the protein of the invention or a homologous protein or a closely related molecule, may be used to identify nucleic acid sequences which encode the respective protein. The hybridization probes of the subject invention may be DNA or RNA and derived from the nucleotide sequence of the polynucleotide encoding the protein of the invention or from a genomic sequence including promoter, enhancer elements, and introns of the naturally occurring gene. Hybridization probes may be labeled by a variety of reporter groups, for example, radionuclides such as ³²P or ³⁵S or enzymatic labels, such as alkaline phosphatase coupled to the probe via avidin/biotin coupling systems, and the like.

Polynucleotide sequences specific for the protein of the invention or homologous nucleic acids may be used for the diagnosis of conditions or diseases, which are associated with the expression of the protein. Examples of such conditions or diseases include, but are not limited to, metabolic diseases and disorders, including obesity and diabetes. Polynucleotide sequences specific for the protein of the invention or a homologous protein may also be used to monitor the progress of patients receiving treatment for metabolic diseases and disorders, including obesity and diabetes. The polynucleotide sequences may be used qualitative or quantitative assays, e.g. in Southern or Northern analysis, dot blot or other membrane-based technologies; in PCR technologies; or in dip stick, pin, ELISA or chip assays utilizing fluids or tissues from patient biopsies to detect altered gene expression. Such qualitative or quantitative methods are well known in the art.

In a particular aspect, the nucleotide sequences specific for the protein of the invention or homologous nucleic acids may be useful in assays that detect activation or induction of various metabolic diseases or dysfunctions, including metabolic syndrome, obesity, and/or diabetes as well as related disorders such as eating disorder, cachexia, hypertension, coronary heart disease, hypercholesterolemia, dyslipidemia, osteoarthritis, gallstones, or liver fibrosis. The nucleotide sequences may be labeled by standard methods, and added to a fluid or tissue sample from a patient under conditions suitable for the formation of hybridization complexes. After a suitable incubation period, the sample is washed and the signal is quantitated and compared with a standard value. If the amount of signal in the biopsied or extracted sample is significantly altered from that of a comparable have hybridized with nucleotide sequences in the sample, and the presence of altered levels of nucleotide sequences encoding the protein of the invention or a homologous protein in the sample indicates the presence of the associated disease. Such assays may also be used to evaluate the efficacy of a particular therapeutic treatment regimen in animal studies, in clinical trials or in monitoring the treatment of an individual patient.

In order to provide a basis for the diagnosis of a disease associated with expression of the protein of the invention or a homologous protein, a normal or standard profile for expression is established. This may be accomplished by combining body fluids or cell extracts taken from normal subjects, either animal or human, with a sequence or a fragment thereof, which is specific for the nucleic acids encoding the protein of the invention or homologous nucleic acids, under conditions suitable for hybridization or amplification. Standard hybridization may be quantified by comparing the values obtained from normal subjects with those from an experiment where a known amount of a substantially purified polynucleotide is used. Standard values obtained from normal samples may be compared with values obtained from samples from patients who are symptomatic for disease. Deviation between standard and subject values is used to establish the presence of disease. Once disease is established and a treatment protocol is initiated, hybridization assays may be repeated on a regular basis to evaluate whether the level of expression in the patient begins to approximate that, which is observed in the normal patient. The results obtained from successive assays may be used to show the efficacy of treatment over a period ranging from several days to months.

With respect to metabolic or pancreatic diseases such as described above the presence of an unusual amount of transcript in biopsied tissue from an individual may indicate a predisposition for the development of the disease or may provide a means for detecting the disease prior to the appearance of actual clinical symptoms. A more definitive diagnosis of this type may allow health professionals to employ preventative measures or aggressive treatment earlier thereby preventing the development or further progression of the metabolic diseases and disorders.

Additional diagnostic uses for oligonucleotides designed from the sequences encoding the protein of the invention or a homologous protein may involve the use of PCR. Such oligomers may be chemically synthesized, generated enzymatically or produced from a recombinant source. Oligomers will preferably consist of two nucleotide sequences, one with sense orientation (5′.fwdarw.3′) and another with antisense (3′.rarw.5′), employed under optimized conditions for identification of a specific gene or condition. The same two oligomers, nested sets of oligomers or even a degenerate pool of oligomers may be employed under less stringent conditions for detectiori and/or quantification of closely related DNA or RNA sequences.

Methods which may also be used to quantitate the expression of the protein of the invention or a homologous protein include radiolabeling or biotinylating nucleotides, coamplification of a control nucleic acid, and standard curves onto which the experimental results are interpolated (Melby P. C. et al., (1993) J. Immunol. Methods, 159: 235-244; Duplaa C. et al., (1993) Anal. Biochem. 212: 229-236). The speed of quantification of multiple samples may be accelerated by running the assay in an ELISA format where the oligomer of interest is presented in various dilutions and a spectrophotometric or colorimetric response gives rapid quantification.

In another embodiment of the invention, the nucleic acid sequences may also be used to generate hybridization probes, which are useful for mapping the naturally occurring genomic sequence. The sequences may be mapped to a particular chromosome or to a specific region of the chromosome using well known techniques. Such techniques include FISH, FACS or artificial chromosome constructions, such as yeast artificial chromosomes, bacterial artificial chromosomes, bacterial P1 constructions or single chromosome cDNA libraries as reviewed in Price C. M., (1993) Blood Rev. 7: 127-134, and Trask B. J., (1991) Trends Genet. 7: 149-154. FISH is described in Verma R. S, and Babu A., (1989) Human Chromosomes: A Manual of Basic Techniques, Pergamon Press, New York, N.Y. The results may be correlated with other physical chromosome mapping techniques and genetic map data. Examples of genetic map data can be found in the 1994 Genome Issue of Science (265:1981f). Correlation between the location of the gene encoding the protein of the invention on a physical chromosomal map and a specific disease or predisposition to a specific disease, may help to delimit the region of DNA associated with that genetic disease.

The nucleotide sequences of the subject invention may be used to detect differences in gene sequences between normal, carrier or affected individuals. For example, an analysis of polymorphisms, e.g. single nucleotide polymorphisms may be carried out. Further, in situ hybridization of chromosomal preparations and physical mapping techniques such as linkage analysis using established chromosomal markers may be used for extending genetic maps. Often the placement of a gene on the chromosome of another mammalian species, such as mouse, may reveal associated markers even if the number or arm of a particular human chromosome is not known. New sequences can be assigned to chromosomal arms or parts thereof, by physical mapping. This provides valuable information to investigators searching for disease genes using positional cloning or other gene discovery techniques. Once the disease or syndrome has been crudely localized by genetic linkage to a particular genomic region, for example, AT to 11q22-23 (Gatti R. A. et al., (1988) Nature 336: 577-580), any sequences mapping to that area may represent associated or regulatory genes for further investigation. The nucleotide sequences of the subject invention may also be used to detect differences in the chromosomal location due to translocation, inversion, etc. among normal, carrier or affected individuals.

In another embodiment of the invention, the protein of the invention, its catalytic or immunogenic fragments or oligopeptides thereof, an in vitro model, a genetically altered cell or animal, can be used for screening libraries of compounds in any of a variety of drug screening techniques. One can identify modulators/effectors, e.g. receptors, ligands or substrates that bind to, modulate or mimic the action of the protein of the invention. The protein or fragment thereof employed in such screening may be free in solution, affixed to a solid support, borne on a cell surface, or located intracellularly. The formation of binding complexes, between the protein of the invention and the agent tested, may be measured. Agents could also, either directly or indirectly, influence the activity of the protein of the invention.

In addition activity of DG147 or homologous proteins against their physiological substrate(s) or derivatives thereof could be measured in cell-based or cell-free assays. Agents may also interfere with posttranslational modifications of the protein of the invention, such as phosphorylation and dephosphorylation, farnesylation, palmitoylation, acetylation, alkylation, ubiquitination, proteolytic processing, subcellular localization or degradation. Moreover, agents could influence the dimerization or oligomerization of the protein of the invention or, in a heterologous manner, of the protein of the invention with other proteins, for example, but not exclusively, docking proteins, enzymes, receptors, ion channels, uncoupling proteins, or translation factors. Agents could also act on the physical interaction of the proteins of this invention with other proteins, which are required for protein function, for example, but not exclusively, their downstream signaling.

Methods for determining protein-protein interaction are well known in the art. For example binding of a fluorescently labeled peptide derived from the protein of the invention to the interacting protein (or vice versa) could be detected by a change in polarisation. In case that both binding partners, which can be either the full length proteins as well as one binding partner as the full length protein and the other just represented as a peptide are fluorescently labeled, binding could be detected by fluorescence energy transfer (FRET) from one fluorophore to the other. In addition, a variety of commercially available assay principles suitable for detection of protein-protein interaction are well known In the art, for example but not exclusively AlphaScreen (PerkinElmer) or Scintillation Proximity Assays (SPA) by Amersham. Alternatively, the interaction of the protein of the invention with cellular proteins could be the basis for a cell-based screening assay, in which both proteins are fluorescently labeled and interaction of both proteins is detected by analysing cotranslocation of both proteins with a cellular imaging reader, as has been developed for example, but not exclusively, by Cellomics or EvotecOAl. In all cases the two or more binding partners can be different proteins with one being the protein of the invention, or in case of dimerization and/or oligomerization the protein of the invention itself. The protein of the invention, for which one target mechanism of interest, but not the only one, would be such protein/protein interaction, is the DG147 protein.

Of particular interest are screening assays for agents that have a low toxicity for mammalian cells. The term “agent” as used herein describes any molecule, e.g. protein or pharmaceutical, with the capability of altering or mimicking the physiological function of the protein of the invention. Candidate agents encompass numerous chemical classes, though typically they are organic molecules, preferably small organic compounds having a molecular weight of more than 50 and less than about 2,500 Daltons. Candidate agents comprise functional groups necessary for structural interaction with proteins, particularly hydrogen bonding, and typically include at least an amine, carbonyl, hydroxyl or carboxyl group, preferably at least two of the functional chemical groups. The candidate agents often comprise carbocyclic or heterocyclic structures and/or aromatic or polyaromatic structures substituted with one or more of the above functional groups.

Candidate agents are also found among biomolecules including peptides, saccharides, fatty acids, steroids, purines, pyrimidines, nucleic acids and derivatives, structural analogs or combinations thereof. Candidate agents are obtained from a wide variety of sources including libraries of synthetic or natural compounds. For example, numerous means are available for random and directed synthesis of a wide variety of organic compounds and biomolecules, including expression of randomized oligonucleotides and oligopeptides. Alternatively, libraries of natural compounds in the form of bacterial, fungal, plant and animal extracts are available or readily produced. Additionally, natural or synthetically produced libraries and compounds are readily modified through conventional chemical, physical and biochemical means, and may be used to produce combinatorial libraries. Known pharmacological agents may be subjected to directed or random chemical modifications, such as acylation, alkylation, esterification, amidification, etc. to produce structural analogs. Where the screening assay is a binding assay, one or more of the molecules may be joined to a label, where the label can directly or indirectly provide a detectable signal.

Another technique for drug screening, which may be used, provides for high throughput screening of compounds having suitable binding affinity to the protein of interest as described in published PCT application WO84/03564. In this method, as applied to the protein of the invention large numbers of different small test compounds, e.g. aptamers, peptides, low-molecular weight compounds etc., are provided or synthesized on a solid substrate, such as plastic pins or some other surface. The test compounds are reacted with the proteins or fragments thereof, and washed. Bound proteins are then detected by methods well known in the art. Purified proteins can also be coated directly onto plates for use in the aforementioned drug screening techniques. Alternatively, non-neutralizing antibodies can be used to capture the peptide and immobilize it on a solid support.

In another embodiment, one may use competitive drug screening assays in which neutralizing antibodies capable of binding the protein specifically compete with a test compound for binding the protein. In this manner, the antibodies can be used to detect the presence of any peptide, which shares one or more antigenic determinants with DG147 or a homologous protein.

The nucleic acids encoding the protein of the invention can be used to generate transgenic animals or site-specific gene modifications in cell lines. These transgenic non-human animals are useful in the study of the function and regulation of said protein in vivo. Transgenic animals, particularly mammalian transgenic animals, can serve as a model system for the investigation of many developmental and cellular processes common to humans. A variety of non-human models of metabolic disorders can be used to test modulators/effectors of the protein of the invention. Misexpression (for example, over-expression or lack of expression) of the protein of the invention, particular feeding conditions, and/or administration of biologically active compounds can create models of metabolic disorders.

In one embodiment of the invention, such assays use mouse models of insulin resistance and/or diabetes, such as mice carrying gene knockouts in the leptin pathway (for example, ob (leptin) or db (leptin receptor) mice). Such mice develop typical symptoms of diabetes, show hepatic lipid accumulation and frequently have increased plasma lipid levels (see Bruning J. C. et al, (1998) Mol. Cell. 2: 559-569). Susceptible wild type mice (for example C57BI/6) show similar symptoms if fed a high fat diet. In addition to testing the expression of the protein of the invention in such mouse strains (see Examples section), these mice could be used to test whether administration of a candidate modulator/effector alters for example lipid accumulation in the liver, in plasma, or adipose tissues using standard assays well known in the art, such as FPLC, calorimetric assays, blood glucose level tests, insulin tolerance tests and others.

Transgenic animals may be made through homologous recombination in non-human embryonic stem cells, where the normal locus of the gene encoding the protein of the invention is altered. Alternatively, a nucleic acid construct encoding the protein of the invention is injected into oocytes and is randomly integrated into the genome. Vectors for stable integration include plasmids, retroviruses and other animal viruses, yeast artificial chromosomes (YACs), and the like. The modified cells or animal are useful in the study of the function and regulation of the protein of the invention. For example, a series of small deletions and/or substitutions may be made in the gene that encodes the protein of the invention to determine the role of particular domains of the protein, functions in pancreatic differentiation, etc.

Furthermore, variants of the gene of the invention like specific constructs of interest include anti-sense molecules, which will block the expression of the protein of the invention, or expression of dominant negative mutations. A detectable marker, such as for example lac-Z or luciferase may be introduced in the locus of the gene of the invention, where up regulation of expression of the gene of the invention will result in an easily detected change in phenotype.

One may also provide for expression of the genes of the invention or variants thereof in cells or tissues where it is not normally expressed or at abnormal times of development. In addition, by providing expression of the protein of the invention in cells in which they are not normally produced, one can induce changes in cell behavior.

DNA constructs for homologous recombination will comprise at least portions of the genes of the invention with the desired genetic modification, and will include regions of homology to the target locus. DNA constructs for random integration do not need to contain regions of homology to mediate recombination. Conveniently, markers for positive and negative selection are included. DNA constructs for random integration will consist of the nucleic acids encoding the protein of the invention, a regulatory element (promoter), an intron and a poly-adenylation signal. Methods for generating cells having targeted gene modifications through homologous recombination are known in the art. For non-human embryonic stem (ES) cells, an ES cell line may be employed, or embryonic cells may be obtained freshly from a host, e.g. mouse, rat, guinea pig, etc. Such cells are grown on an appropriate fibroblast-feeder layer and are grown in the presence of leukemia inhibiting factor (LIF).

When non-human ES or embryonic cells or somatic pluripotent stem cells have been transfected, they may be used to produce transgenic animals. After transfection, the cells are plated onto a feeder layer in an appropriate medium. Cells containing the construct may be selected by employing a selective medium. After sufficient time for colonies to grow, they are picked and analyzed for the occurrence of homologous recombination or integration of the construct. Those colonies that are positive may then be used for embryo transfection and morula aggregation. Briefly, morulae are obtained from 4 to 6 week old superovulated females, the Zona Pellucida is removed and the morulae are put into small depressions of a tissue culture dish. The ES cells are trypsinized, and the modified cells are placed into the depression closely to the morulae. On the following day the aggregates are transferred into the uterine horns of pseudo-pregnant females. Females are then allowed to go to term. Chimeric offsprings can be readily detected by a change in coat color and are subsequently screened for the transmission of the mutation into the next generation (F1-generation). Offspring of the F1-generation are screened for the presence of the modified gene and males and females having the modification are mated to produce homozygous progeny. If the gene alterations cause lethality at some point in development, tissues or organs can be maintained as allogenic or congenic grafts or transplants, or in vitro culture. The transgenic animals may be any non-human mammal, such as laboratory animal, domestic animals, etc., for example, mouse, rat, guinea pig, sheep, cow, pig, and others. The transgenic animals may be used in functional studies, drug screening, and other applications and are useful in the study of the function and regulation of the protein of the invention in vivo.

Finally, the invention also relates to a kit comprising at least one of
(a) a nucleic acid molecule coding for the protein of the invention or a functional fragment thereof;
(b) the protein of the invention or a functional fragment or an isoform thereof;
(c) a vector comprising the nucleic acid of (a);
(d) a host cell comprising the nucleic acid of (a) or the vector of (c);
(e) a polypeptide encoded by the nucleic acid of (a);
(f) a fusion polypeptide encoded by the nucleic acid of (a);
(g) an antibody, an aptamer or another modulator/effector of the nucleic acid of (a) or the polypeptide of (b), (e), or (f) and
(h) an anti-sense oligonucleotide of the nucleic acid of (a).

The kit may be used for diagnostic or therapeutic purposes or for screening applications as described above. The kit may further contain user instructions.

The Figures show:

FIG. 1 shows the real-time PCR analysis of DG147 expression in mammalian (mouse) tissues.

FIG. 1A shows the analysis of DG147 expression in wild type (referred to as wt-mice) and control diet (referred to as controldiet) mouse tissues.

FIG. 1B shows the analysis of DG147 expression in a mouse model used to study metabolic disorders (referred to as oblob-mice) compared to wild type mice and in mice fed with a high fat diet (referred to as HFD-mice) compared to mice fed with a control diet.

FIG. 2 shows the expression of DG147 in human adipose tissue. Shown is the microarray analysis of DG147 expression in human abdominal derived primary adipocyte cells during the differentiation from preadipocytes to mature adipocytes.

FIG. 3 shows in situ hybridization results for the DG147 protein of the invention.

FIG. 3A shows a mouse embryo (day E11.5; lateral view) stained with a whole-mount in situ hybridization method, the left body wall is removed.

FIG. 3B shows the isolated inner organs (lateral view) isolated from a mouse embryo (day E11.5) stained with a whole-mount in situ hybridization method.

The examples illustrate the invention:

### EXAMPLE 1

### Identification of Secreted Factors Expressed in Pancreas

A screen for secreted factors expressed in developing mouse pancreas was carried out according to methods known by those skilled in the art (see, for example Pera E. M. and De Robertis E. M., (2000) Mech Dev 96(2): 183-195) with several modifications.

### Expression cDNA Library:

During organogenesis, the pancreatic bud is surrounded and influenced by the associated mesenchyme. (see for example, Madsen O. D. et al., (1996) Eur. J. Biochem. 242:435-445 and Slack, J. M., (1995) Development 121:1569-1580). Recently, it was suggested, that white adipocytes origin directly from mesenchymal cells (Atanossova P. K, (2003) Folia Med. 45: 41-45). During embryogenesis, the innervation and vascularization of the pancreas can be observed. Therefore, the tissue used in the screen might have contained besides pancreatic cells some adipocyte precursors, blood vessels, as well as neuronal cells.

A mouse embryonic stage 9.5-15 pancreatic bud library was prepared in pCMVSPORT-6 vector using SUPERSCRIPT Plasmid System from Invitrogen according to the manufacturer's instructions. The non-amplified library was electroporated into MaxEff DH10B cells (Invitrogen).

### Secretion Cloning

Bacterial clones were picked with sterile toothpicks from agar plates and cultured in 96-deep-well microtiter plates in LB-ampicillin (see Sambrook et al., supra). Aliquots of 8 cultures were pooled, and plasmid DNA was isolated using the BioRobot_{—}9600 apparatus according to the manufacturer's instructions (Qiagen; QIAprep(r) Turbo BioRobot Kit. Human 293 cell culture cells were cultured in 75 ml tissue culture flasks in DMEM and 10% fetal calf serum. At 90-99% confluence, the cells were splitted at 1:3 ratio and plated onto poly-D-lysine (Sigma) coated 96-well plates. Cells were transfected with 100-500 ng plasmid using lipofectamine 2000 (Invitrogen). After 6 hours, the medium was exchanged for fresh complete growth medium. 24 hours after transfection, the cells were washed twice with DMEM without cysteine and methionine (Invitrogen), supplemented with 1% dialysed bovine serum (Sigma) with 50 μg/ml per ml Heparin (Sigma) and glutamine. The cells were labeled radioactively (‘S35 Met-label’, from Hartmann Analytic GmbH). After 12 hours, aliquots of the supernatants were harvested in 96-well PCR plates and subjected to SDS gel electrophoresis in precast 4-20% gradient polyacrylamide Criterion gels (Biorad) under reducing conditions, using Criterion Dodeca Cell gel running chamber (Biorad). The gels were fixed in 10% acetic acid, 25% isopropanol for 30 min, soaked 15-30 min in AMPLIFY reagent (Amersham), dried and exposed to X-OMAT (AR) film (Kodak). Positive clones were identified and regrown in 96-well-plates. DNA of individual clones was prepared and used for transfection as described above. If one of the clones yielded proteins of the same size as that of the original pool, a positive clone was identified. Positive clones were partially sequenced from the 5′ end (SEQLAB, Goettingen).

### EXAMPLE 2

### Identification of the Human Homologous Nucleic Acid and Protein Sequences

The term “polynucleotide comprising the nucleotide sequence as shown in GenBank Accession number” relates to the expressible gene of the nucleotide sequences deposited under the corresponding GenBank Accession number. The term “GenBank Accession number” relates to NCBI GenBank database entries (Ref.: Benson D. A. et al., (2000) Nucleic Acids Res. 28: 15-18).

Human DG147 relates to GenBank Accession Number NM_{—}001831 (nucleic acid) and the protein encoded by this sequence, e.g. GenBank Accession number NP_{—}001822 (protein). Mouse DG147 relates to GenBank Accession Number NM_{—}013492 for the cDNA and NP_{—}038520 for the protein.

Further, DG147 or homologous proteins and nucleic acid molecules coding therefore are obtainable from other vertebrate species, e.g. mammals or birds.

### EXAMPLE 3

### Expression of the DG147 Polypeptide in Mouse Tissues—TaqMan Analysis

To analyse the expression of the DG147 mRNA disclosed in this invention in mammalian tissues, several mouse strains (preferably mice strains C57BI/6J, C57BI/6 ob/ob, C57BI/KS db/db, and Non-Obese-Diabetic (NOD) mice, which are standard model systems in obesity and diabetes research) were purchased from Harlan Winkelmann (33178 Borchen, Germany) and Taconic M & B (Germantown, N.Y. 12526, U.S.A.), respectively, and maintained under constant temperature (preferably 22° C.), 40 percent humidity and a light/dark cycle of preferably 14/10 hours. The mice were fed a standard chow (for example, from ssniff Spezialitaten GmbH, order number ssniff M-Z V1126-000). For the fasting experiment (“fasted wild type mice”), wild type mice were starved for 48 h without food, but only water supplied ad libitum. (see, for example, Schnetzler et al. J Clin Invest 1993 July; 92(1):272-80, Mizuno et al. Proc Natl Acad Sci USA 1996 Apr. 16; 93(8): 3434-8). In a further experiment wild-type (wt) mice were fed a control diet (preferably Altromin C1057mod control, 4.5% crude fat) or high fat diet (preferably Altromin C1057mod. high fat, 23.5% crude fat). Animals were sacrificed at an age of 6 to 8 weeks. The animal tissues were isolated according to standard procedures known to those skilled in the art, snap frozen in liquid nitrogen and stored at −80° C. until needed.

RNA was isolated from mouse tissues using Trizol Reagent (for example, from Invitrogen, Karlsruhe, Germany) and further purified with the RNeasy Kit (for example, from Qiagen, Germany) in combination with an DNase-treatment according to the instructions of the manufacturers and as known to those skilled in the art. Total RNA was reverse transcribed (preferably using Superscript II RNaseH⁻ Reverse Transcriptase, from Invitrogen, Karlsruhe, Germany) and subjected to Taqman analysis preferably using the Taqman 2xPCR Master Mix (from Applied Biosystems, Weiterstadt, Germany; the Mix contains according to the Manufacturer for example AmpliTaq Gold DNA Polymerase, AmpErase UNG, dNTPs with dUTP, passive reference Rox and optimized buffer components) on a GeneAmp 5700 Sequence Detection System (from Applied Biosystems, Weiterstadt, Germany).

The following prime/probe pair was used for the TaqMan analysis (GenBank Accession Number NM_{—}013492 for the mouse DG147 sequence):

Mouse DG147 forward primer (Seq ID NO:1): 5′-TGG AGA GTG ACC GGC AGC—3′; mouse DG147 reverse primer (Seq ID NO:2): 5′-GCC AGA TGC CCG AGC A-3′; mouse DG147 Taqman probe (Seq ID NO:3): (5/6-FAM)-CCT GGA TGC CAT GCA GGA CAG CT-(5/6-TAMRA).

In FIG. 1 the relative RNA-expression is shown on the Y-axis and the tissues tested are given on the X-axis. “WAT” refers to white adipose tissue. The panel of the wild type mice tissues comprises liver, pancreas, muscle, small intestine, WAT, hypothalamus, and heart, and the panel of the control diet-mice tissues comprises liver, muscle, small intestine, WAT, brain, and heart.

The function of the DG147 protein in metabolism was further validated by analyzing the expression of the transcript in different tissues. For this passage, mouse models of insulin resistance and/or diabetes were used, such as mice carrying gene knockouts in the leptin pathway (for example, ob/ob (leptin) or db/db (leptin receptor/ligand) mice) to study the expression of the proteins of the invention. Such mice develop typical symptoms of diabetes, show hepatic lipid accumulation and frequently have increased plasma lipid levels (see Bruning J. C. et al, (1998) Mol. Cell. 2: 559-569).

Further, expression of the mRNAs encoding the proteins of the invention was also examined in susceptible wild type mice (for example, C57BI/6) that show symptoms of diabetes, lipid accumulation, and high plasma lipid levels, if fed a high fat diet.

Expression profiling studies confirm the particular relevance of the DG147 protein as regulator of energy metabolism in mammals.

Taqman analysis revealed that DG147 is expressed in several mammalian tissues, showing highest level of expression in white adipose tissue (WAT), and lower but still high levels in further tissues, e.g. liver, hypothalamus, brain, heart, pancreas, musde, and small intestine, as shown in FIG. 1A. We found, for example, that the expression of DG147 is dramatically down-regulated in the WAT of ob/ob mice compared to wild-type mice, as well as in wild type mice fed a high fat diet (see FIG. 1B). The outstanding high expression of DG147 in WAT of wild type and control mice suggest that it plays an essential role in adipocyte metabolism. The strong down regulation of DG147 expression in the mouse models for the metabolic syndrome as described above suggests that it also might play a role in the regulation of energy homeostasis.

### EXAMPLE 4

### Analysis of the Differential Expression of the DG147 Transcript in Human Tissues

RNA preparation from human primary adipose tissues was done as described in Example 3. The target preparation, hybridization, and scanning was performed as described in the manufactures manual (see Affymetrix Technical Manual, 2002, obtained from Affinetrix, Santa Clara, USA).

The expression analysis (using Affymetrix GeneChips) of the genes using primary human abdominal adipocycte differentiation clearly shows differential expression of the human DG147 gene in adipocytes. Several independent experiments were done. In FIG. 2, the fluorescent intensity is shown on the Y-axis, and the X-axis represents the time axis. “d 0” refers to day 0 (start of the experiment), “d12” refers to day 12 of adipocyte differentiation.

The experiments show that the DG147 transcript is most abundant at day 12 compared to day 0 during differentiation (see FIG. 2). Thus, the DG147 protein has to be increased in order for the preadipocyctes to differentate into mature adipocyctes. The DG147 protein in preadipocyctes has the potential to enhance adipose differentiation. Therefore, the DG147 protein might play an essential role in the regulation of human metabolism, in particular in the regulation of adipogenesis and thus it might be an essential role in pancreatic diseases (e.g. diabetes), obesity, and/or metabolic syndrome.

### EXAMPLE 5

### In situ Hybridisations

Whole-mount in situ hybridizations were performed according to standard protocols as known to those skilled in the art and as described previously (for example, Pelton, R. W. et al., (1990) Development 110, 609-620; Belo, J. A. et al., (1997) Mech. Dev. 68, 45-57).

The nucleic acid sequence encoding the mouse DG147 protein is expressed in the internal organs including the gastrointestinal tract (see FIG. 3).

For the purpose of the present invention, it will be understood by the person having average skill in the art, that any combination of any feature mentioned throughout the specification is explicitly disclosed herewith.

1. A pharmaceutical composition comprising a DG147 protein or/and a functional fragment thereof, a nucleic acid molecule encoding a DG147 protein or/and a functional fragment thereof or/and a modulator/effector of said nucleic acid molecule or/and said protein together with pharmaceutically acceptable carriers, diluents or/and additives.

2. The composition of claim 1, wherein the nucleic acid molecule is a vertebrate DG147 nucleic acid, particularly encoding a human DG147 protein as described in Example 2 or/and a nucleic molecule which is complementary thereto or a functional fragment thereof or a variant thereof.

3. The composition of claim 1, wherein said DG147 nucleic acid molecule is selected from the group consisting of
(a) a nucleic acid molecule encoding a polypeptide, particularly as described in Example 2 or an isoform, fragment or variant of said polypeptide;
(b) a nucleic acid molecule which comprises or is the nucleic acid molecule as described in Example 2;
(c) a nucleic acid molecule being degenerated as a result of the genetic code to the nucleic acid sequences as defined in (a) or (b);
(d) a nucleic acid molecule that hybridizes at 50° C. in a solution containing 1×SSC and 0.1% SDS to a nucleic acid molecule as defined in claim 2 or as defined in (a) to (c) or/and a nucleic acid molecule which is complementary thereto;
(e) a nucleic acid molecule that encodes a polypeptide which is at least 85%, preferably at least 90%, more preferably at least 95%, more preferably at least 98% and up to 99.6% identical to the human protein as described in Example 2 or as defined in claim 2 or to a polypeptide as defined in (a);
(f) a nucleic acid molecule that differs from the nucleic acid molecule of (a) to (e) by mutation and wherein said mutation causes an alteration, deletion, duplication or premature stop in the encoded polypeptide.

4. The composition of claim 1, wherein the nucleic acid molecule is a DNA molecule, particularly a cDNA or a genomic DNA.

5. The composition of claim 1, wherein said nucleic acid encodes a polypeptide contributing to regulating the energy homeostasis.

6. The composition of claim 1, wherein said nucleic acid encodes a polypeptide contributing to the differentiation of adipocytes.

7. The composition of claim 1, wherein said nucleic acid molecule is a recombinant nucleic acid molecule.

8. The composition of claim 1, wherein the nucleic acid molecule is a vector, particularly an expression vector.

9. The composition of claim 1, wherein the polypeptide is a recombinant polypeptide.

10. The composition of claim 9, wherein said recombinant polypeptide is a fusion polypeptide.

11. The composition of claim 1, wherein said nucleic acid molecule is selected from hybridization probes, primers and anti-sense oligonucleotides.

12. The composition of claim 1 which is a diagnostic composition.

13. The composition of claim 1 which is a therapeutic composition.

14. The composition of claim 1 for the manufacture of an agent for detecting or/and verifying, for the treatment, alleviation or/and prevention of metabolic diseases or dysfunctions, including metabolic syndrome, obesity, or/and diabetes, as well as related disorders such as eating disorder, cachexia, hypertension, coronary heart disease, hypercholesterolemia, dyslipidemia, osteoarthritis, gallstones, or liver fibrosis, in cells, cell masses, organs or/and subjects.

15. The composition of claim 1 for the manufacture of an agent for the regulation of adipogenesis, particularly for the regulation of adipocyte differentiation.

16. The composition of claim 1 for application in vivo.

17. The composition of claim 1 for application in vitro.

18. Use of a nucleic acid molecule encoding a DG147 protein or an isoform, a functional fragment or variant thereof, in particular a nucleic acid molecule as described in Example 2, particularly of a nucleic acid molecule according to claim 3 (a), (b), or (c), or/and a polypeptide encoded thereby or/and a functional fragment or/and a variant of said nucleic acid molecule or said polypeptide or/and a modulator/effector of said nucleic acid molecule or polypeptide for the manufacture of a medicament for the treatment of obesity, diabetes, or/and metabolic syndrome for controlling the function of a gene or/and a gene product which is influenced or/and modified by a DG147 polypeptide, particularly by a polypeptide according to claim 3.

19. Use of a nucleic acid molecule encoding a DG147 protein or an isoform, a functional fragment or variant thereof, in particular a nucleic acid molecule as described in Example 2, particularly of a nucleic acid molecule according to claim 3 (a), (b), or (c), or/and a polypeptide encoded thereby or/and a functional fragment or/and a variant of said nucleic acid molecule or said polypeptide or/and a modulator/effector of said nucleic acid molecule or said polypeptide for identifying substances capable of interacting with a DG147 polypeptide, particularly with a polypeptide according to claim 3.

20. A non-human transgenic animal exhibiting a modified expression of a DG147 polypeptide, particularly of a polypeptide according to claim 3.

21. The animal of claim 20, wherein the expression of the DG147 polypeptide, particularly of a polypeptide according to claim 3, is increased or/and reduced.

22. A recombinant host cell exhibiting a modified expression of a DG147 polypeptide, particularly of a polypeptide according to claim 3.

23. The cell of claim 22 which is a human cell.

24. A method of identifying a (poly)peptide involved in the regulation of energy homeostasis or/and differentiation of adipocytes in a mammal comprising the steps of
(a) contacting a collection of (poly)peptides with a DG147 polypeptide, particularly a polypeptide according to claim 3, or a functional fragment thereof under conditions that allow binding of said (poly)peptides;
(b) removing (poly)peptides which do not bind and
(c) identifying (poly)peptides that bind to said DG147 polypeptide.

25. A method of screening for an agent which modulates/effects the interaction of a DG147 polypeptide, particularly of a polypeptide according to claim 3, with a binding target, comprising the steps of
(a) incubating a mixture comprising
(aa) a DG147 polypeptide, particularly a polypeptide according 5 to claim 3, or a functional fragment thereof;
(ab) a binding target/agent of said polypeptide or functional fragment thereof; and
(ac) a candidate agent under conditions whereby said polypeptide or functional fragment thereof specifically binds to said binding target/agent at a reference affinity;
(b) detecting the binding affinity of said polypeptide or functional fragment thereof to said binding target to determine an affinity for the agent; and
(c) determining a difference between affinity for the agent and the reference affinity.

26. A method for screening for an agent, which modulates/effects the activity of a DG147 polypeptide, particularly of a polypeptide according to claim 3, comprising the steps of
(a) incubating a mixture comprising
(aa) said polypeptide or a functional fragment thereof; (ab) a candidate agent under conditions whereby said polypeptide or functional fragment thereof has a reference activity;
(b) detecting the activity of said polypeptide or functional fragment thereof to determine an activity in presence of the agent; and
(c) determining a difference between the activity in the presence of the agent and the reference activity.

27. A method of producing a composition comprising the (poly)peptide identified by the method of claim 24 with a pharmaceutically acceptable carrier, diluent or/and additive.

28. The method of claim 27 wherein said composition is a pharmaceutical composition for preventing, alleviating or treating of metabolic diseases or dysfunctions, including obesity, diabetes, or/and metabolic syndrome, as well as related disorders such as eating disorder, cachexia, hypertension, coronary heart disease, hypercholesterolemia, dyslipidemia, osteoarthritis, gallstones, or liver fibrosis.

29. Use of a (poly)peptide as identified by the method of claim 24 for the preparation of a pharmaceutical composition for the treatment, alleviation or/and prevention of metabolic diseases or dysfunctions, including obesity, diabetes, or/and metabolic syndrome, as well as related disorders such as eating disorder, cachexia, hypertension, coronary heart disease, hypercholesterolemia, dyslipidemia, osteoarthritis, gallstones, or liver fibrosis.

30. Use of a nucleic acid molecule as defined in claim 1, for the preparation of a medicament for the treatment, alleviation or/and prevention of metabolic diseases or dysfunctions, including obesity, diabetes, or/and metabolic syndrome, as well as related disorders such as eating disorder, cachexia, hypertension, coronary heart disease, hypercholesterolemia, dyslipidemia, osteoarthritis, gallstones, or liver fibrosis.

31. Use of a polypeptide as defined in claim 1 for the preparation of a medicament for the treatment, alleviation or/and prevention of metabolic diseases or dysfunctions, including obesity, diabetes, or/and metabolic syndrome, as well as related disorders such as eating disorder, cachexia, hypertension, coronary heart disease, hypercholesterolemia, dyslipidemia, osteoarthritis, gallstones, or liver fibrosis.

32. Use of a vector as defined in claim 8 for the preparation of a medicament for the treatment, alleviation or/and prevention of metabolic diseases or dysfunctions, including obesity, diabetes, or/and metabolic syndrome, as well as related disorders such as eating disorder, cachexia, hypertension, coronary heart disease, hypercholesterolemia, dyslipidemia, osteoarthritis, gallstones, or liver fibrosis.

33. Use of a host cell as defined in claim 22 for the preparation of a medicament for the treatment, alleviation or/and prevention of metabolic diseases or dysfunctions, including obesity, diabetes, or/and metabolic syndrome, as well as related disorders such as eating disorder, cachexia, hypertension, coronary heart disease, hypercholesterolemia, dyslipidemia, osteoarthritis, gallstones, or liver fibrosis.

34. Use of a DG147 nucleic acid molecule or of a functional fragment thereof for the production of a non-human transgenic animal which over- or under-expresses the DG147 gene product.

35. Kit comprising at least one of
(a) a DG147 nucleic acid molecule or a functional fragment thereof;
(b) a DG147 amino acid molecule or a functional fragment or an isoform thereof;
(c) a vector comprising the nucleic acid of (a);
(d) a host cell comprising the nucleic acid of (a) or the vector of (c);
(e) a polypeptide encoded by the nucleic acid of (a);
(f) a fusion polypeptide encoded by the nucleic acid of (a);
(g) an antibody, an aptamer or/and another modulator/effector of the nucleic acid of (a) or/and the polypeptide of (b), (e), or/and (f) and
(h) an anti-sense oligonucleotide of the nucleic acid of (a).

36. A method of producing a composition comprising the agent identified by the method of claim 25 with a pharmaceutically acceptable carrier, diluent or/and additive.

37. A method of producing a composition comprising the agent identified by the method of claim 26 with a pharmaceutically acceptable carrier, diluent or/and additive.

38. Use of an agent as identified by the method of claim 25 for the preparation of a pharmaceutical composition for the treatment, alleviation or/and prevention of metabolic diseases or dysfunctions, including obesity, diabetes, or/and metabolic syndrome, as well as related disorders such as eating disorder, cachexia, hypertension, coronary heart disease, hypercholesterolemia, dyslipidemia, osteoarthritis, gallstones, or liver fibrosis.

39. Use of an agent as identified by the method of claim 26 for the preparation of a pharmaceutical composition for the treatment, alleviation or/and prevention of metabolic diseases or dysfunctions, including obesity, diabetes, or/and metabolic syndrome, as well as related disorders such as eating disorder, cachexia, hypertension, coronary heart disease, hypercholesterolemia, dyslipidemia, osteoarthritis, gallstones, or liver fibrosis.
